(19) United States
(12) Patent Application Publication
(10) Pub. No.: US 2004/0120932 A1
(43) Pub. Date: Jun. 24, 2004

(54) In vitro-derived adult pluripotent stem cells and uses therefor

(76) Inventor: Joseph Edward Zahner, Saint Louis, MO (US)

Correspondence Address: Joseph E. Zahner, Suite C110, 3556 Caroline Mall, St. Louis, MO, 63104-1085, US

(21) Appl. No.: 10/714,211

(22) Filed: Nov. 14, 2003

Related U.S. Application Data
(63) Continuation of application No. 09/919,298, filed on Jul. 31, 2001.
(60) Provisional application No. 60/254,551, filed on Dec. 12, 2000.

(51) Int. Cl. A61K 45/00; A61K 31/7072; A61K 31/19; C12N 5/08

(52) U.S. Cl. 424/93.7; 435/69.1; 435/366; 514/50; 514/575

(57) ABSTRACT

Methods for deriving adult pluripotent stem cells from fully differentiated adult somatic cells by in vitro nuclear remodeling are provided. Cells cultured from a variety of tissue sources are treated in vitro to reverse the tissue specific epigenetic chromosomal changes associated with differentiation. Remodeled cells resemble embryonic stem cells by expressing telomerase and demonstrating pluripotency. The cells can be genetically modified to produce heterologous proteins or to correct for genetic defects. Methods for treating a human by implanting in vitro-derived adult pluripotent stem cells (“NUCREM™ cells”) and generating engineered tissues for implantation are also disclosed. Advantages to this invention include the non-use of embryos to obtain an unlimited supply of stem cells for therapy and the ability to generate autologous cells and tissues for therapeutic use.

This application is a Continuation of U.S. patent application Ser. No. 09/919,298, filed Jul. 31, 2001, which claims priority to Provisional Patent Application No. 60/254,551, filed on Dec. 12, 2000, which is now abandoned.

### SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821 (f).

### BACKGROUND OF THE INVENTION

1. Field of the Invention

This invention relates to the methods and compositions for the production and derivation of human pluripotent stem cell lines from adult somatic cells and therapeutic uses therefor.

2. Description of Related Art

Pluripotent stem cells are self-renewing cells which are capable of differentiating into any one of more than 200 different cell types found in the body. The gold standard of determining pluripotency of a given cell is the ability of that cell to give rise to a complete individual. However, ethical rules prohibit the production of human embryos from stem cells, so other criteria are used to demonstrate the pluripotency of human stem cells. For example, it is readily appreciated by practitioners skilled in the art that human pluripotent stem cells are immortal, may form embryoid bodies containing multiple cell types, may express several embryo specific molecular markers, may give rise to teratomas containing multiple cell types, and may differentiate into mature cell types.

Human pluripotent stem cells are classified in the art as either embryonal carcinoma (“EC”) cells, embryonic germ (“EG”) cells, embryonic stem (“ES”) cells, and adult stem cells. Pluripotent embryonic germ (“EG”) cells are derived from primordial germ cells cultured from 5-9 week old human fetuses (Shamblott, M. J., et al., 1998, Gearhart et al., 2000, 6,090,622). The pluripotency of EG cells are demonstrated by virtue of the fact that they express alkaline phosphatase and the stage-specific embryonic antigens SSEA-1, SSEA-3, SSEA-4, TRA-1-60, TRA-1-81; are passaged continuously while maintaining a normal karyotype; and give rise to embryoid bodies which contain a wide variety of cell types derived from all three primordial germ layers (ectoderm, mesoderm, endoderm).

ES cells were derived from the inner cell mass cells of donated or discarded human blastocyst stage embryos (Thomson, J. A., et al., 1998 and Reubinoff, B. E., et al., 2000). Thomson and coworkers (supra) demonstrated that these human ES cells maintain a normal karyotype after continuous culture; express high levels of telomerase, which is indicative of immortality; express the embryonic markers alkaline phosphatase, SSEA-3, SSEA-4, TRA-1-60, and TRA-1-81; produce teratomas comprised of cells derived of all three germ layers; and are capable of differentiating into other types of cells under suboptimal culture conditions.

In another study, Reubinoff et al. (2000) produced human pluripotent ES cell lines which are immortal and maintain a normal karyotype; express alkaline phosphatase, SSEA-4 and TRA-1-60; produce teratomas in an immunocompromised mouse model, and differentiate under suboptimal growth conditions. Additionally, these ES cells were shown to express the Oct-4 gene, which is an embryonic transcription factor required to maintain cell pluripotency (Nichols, J., et al., 1998 and Hansis, C, et al., 2000). Interestingly, neuronal precursor cells spontaneously differentiated from cultures of these ES cells. Upon subsequent treatment with retinoic acid, these neuronal precursors are able to differentiate into mature neurons. Oct-4 expression is down regulated in differentiated cells.

In addition to pluripotent stem cells of embryonic origin, several groups describe mammalian multipotent stem cell populations that are obtained from adult somatic cell sources. Non-embryonic multipotent stem cells include, for example, neural stem cells, mesenchymal stem cells, bone marrow stem cells and stem cells obtained from liposuction (Zuk et al., 2001). It is important to note that the adult multipotent stem cells described in the prior art have limited potential, in that they have not been demonstrated to give rise to any and all cell types of the body.

Neural stem cells are loosely described as cells which are derived from the nervous system, have the capacity for self-renewal, and can give rise to neural cell types including neurons, astrocytes, and oligodendrocytes (reviewed in Gage, F. H., 2000). Neural stem cells may be obtained from multiple sources within the mammalian brain, including the subventricular zone, hippocampus, ependymal cells, or subgranular zone of the dentate gyrus in mice and rats (reviewed in Gage, 2000 and Clarke, D. L., et al., 2000), the olfactory bulb of adult human patients (Pagano, S. F., 2000), or the forebrain of human embryos (Carpenter, 2000 [U.S. Pat. No. 6,103,530]). In addition to giving rise to neural cell derivatives, neural stem cells also have the capacity to differentiate into other non-neural tissues such as blood cells, for example (Bjornson et al., 1999). Importantly, Clarke et al. (supra) demonstrate that mouse neural stem cells, when cultured in an embryonic environment, can differentiate into a few derivatives of each of the three germ layers, indicating wide multipotentiality.

Mesenchymal stem cells are adult multipotent cells derived from multiple sources, including bone marrow stroma, blood, dermis, and periosteum (Bruder et al., 1998). These cells can be cultured continuously in vitro without spontaneous differentiation. However, under the proper conditions, mesenchymal stem cells can be induced to differentiate into cells of the mesenchymal lineage, including adipocytes, chondrocytes, osteocytes, tenocytes, ligamentogenic cells, myogenic cells, bone marrow stroma cells, and dermogenic cells (Pittenger et al., 1999, and Bruder et al., 1998 [U.S. Pat. No. 5,736,396]). Additionally, mesenchymal cells, upon injection into either mouse or rat brains, are capable of migrating through the brain, engrafting, surviving, and differentiating into astrocytes, ependymal cells, or neurons, suggesting the capacity of mesenchymal stem cells to give rise to cells of a non-mesenchymal lineage (Kopen et al., 1999; Azizi et al., 1998; Caplan and Haynesworth, 1993 [U.S. Pat. No. 5,197,985 and 5,226,914], 1996 [U.S. Pat. No. 5,486,359]; Bruder et al., 1998 [U.S. Pat. No. 5,736,396]). However, it has never been demonstrated or suggested that mesenchymal stem cells can give rise to any cell types of the body.

Hematopoietic stem cells are multipotent cells capable of self renewal and differentiation into multiple blood cells types, including erythrocytes, megakaryocytes, monocytes/macrophages, granulocytes, mast cells, B-cells and T-cells. Hematopoietic stem cells can be obtained from fetal liver, adult bone marrow (Phillips, et al., 2000), or mononuclear muscle precursor cells called satellite cells (Jackson et al., 1999, reviewed in Lemischka, 1999, Tsukamoto et al, 1999 [U.S. Pat. No. 5,914,108], Scadden, 1998 [U.S. Pat. No. 5,827,742], Wagner et al, 1998 [U.S. Pat. No. 5,807,686] and references cited therein.)

It has been proposed that human pluripotent stem cells can be derived via the reprogramming of somatic cell nuclei via nuclear transfer to oocytes (Munsie, et al., 2000). Such an approach, called therapeutic cloning, would allow for pluripotent stem cells derived from the patient to be used in autologous transplant therapy (see Stice, et al., 1999 and 2001). In their paper, Munsie and coworkers established a mouse embryonic stem cell line from embryos generated from cumulus cell nuclei transferred to enucleated oocytes. Although this approach has the advantage of producing cells with unlimited pluripotency, which will not be rejected upon implantation, ethical rules prohibit the cloning of humans or the creation of human embryos for the expressed purpose of establishing embryonic stem cells. However, pluripotent stem cells have never been created by dedifferentiation of adult (non-embryonic, non-fetal) mammalian tissues, and a method for producing such cells has never been demonstrated to date.

Consequently, in the interest of the health of the public there is a great need for methods to produce an unlimited and continuous supply of autologous pluripotent stem cells from sources other than blastocysts, embryos or aborted fetuses. Such a method would be useful for example, for the development of autologous therapeutic cells and engineered tissues for transplantation into patients, thus avoiding the problem of immune system mediated rejection. Furthermore, the production of a potentially unlimited supply of stem cells is a marked improvement over “panning” for adult stem cells (i.e., the detection and purification of stem cells present in an individual through the use of antibodies specific for cell surface antigens), wherein it is very difficult to obtain sufficient numbers of cells for effective therapeutic use.

### References Cited and other Related Art

Patents

Anderson et al., Mammalian multipotent neural stem cells. U.S. Pat. No. 5,928,947 [Jul. 27, 1999].

Anderson, et al., Methods for differentiating neural stem cells to neurons or smooth muscle cells using TGF-beta super family growth factors. U.S. Pat. No. 6,001,654 [Dec. 14, 1999].

Bruder et al. Lineage-directed induction of human mesenchymal stem cell differentiation. U.S. Pat. No. 5,736,396 [Apr. 7, 1998].

Caplan, A. I. and , S. E. Haynesworth, “Method for enhancing the implantation and differentiation of marrow-derived mesenchymal cells.” U.S. Pat. No. 5,197,985 [Mar. 30, 1993].

Caplan, A. I. and , S. E. Haynesworth, “Method for treating connective tissue disorders.” U.S. Pat. No. 5,226,914 [Jul. 13, 1993].

Caplan, A. I. and , S. E. Haynesworth, “Human mesenchymal stem cells.” U.S. Pat. No. 5,486,359 [Jan. 23, 1996].

Carpenter, Cultures of human CNS neural stem cells. U.S. Pat. No. 6,103,530 [Aug. 15, 2000].

Fei, et al., Methods and device for culturing human hematopoietic cells and their precursors. U.S. Pat. No. 5,635,387, Jun. 3, 1997.

Gay, Method of isolating a lineage specific stem cell in vitro. U.S. Pat. No. 5,639,618, Jun. 17, 1997.

Gearhart, et al., Human embryonic pluripotent germ cells. U.S. Pat. No. 6,090,622, Jul. 18, 2000.

Gearhart, et al., Human embryonic germ cell line and methods of use. U.S. Pat. No. 6,245,566, Jun. 12, 2001.

Hogan, Pluripotential embryonic stem cells and methods of making same. U.S. Pat. No. 5,453,357, Sept. 26, 1995.

Johe, Isolation, propagation, and directed differentiation of stem cells from embryonic and adult central nervous system of mammals. U.S. Pat. No. 5,753,506, May 19, 1998.

Keller, et al., Embryonic cell populations and methods to isolate such populations, U.S. Pat. No. 5,914,268 [Jun. 22, 1999].

Quesenberry, Cell compositions for use in transplantation. U.S. Pat. No. 6,068,836 [May 30, 2000].

Scadden, Method of selecting pluripotent hematopoietic progenitor cells. U.S. Pat. No. 5,827,742 [Oct. 27, 1998].

Stice, et al., Cloning using donor nuclei from proliferating somatic cells. U.S. Pat. No. 5,945,577 [Aug. 31, 1999].

Stice et al., CICM cells and non-human mammalian embryos prepared by nuclear transfer of a proliferating differentiated cell or its nucleus. U.S. Pat. No. 6,235,970 [May 22, 2001].

Thomson, Primate embryonic stem cells. U.S. Pat. No. 5,843,780 [Dec. 1, 1998].

Tsukamoto, et al., Human hematopoietic stem cell. U.S. Pat. No. 5,914,108 [Jun. 22, 1999].

Wagner, et al., Pluripotent quiescent stem cell population. U.S. Pat. No. 5,807,686 [Sept. 15, 1998].

Weiss, et al., In vitro growth and proliferation of genetically modified multipotent neural stem cells and their progeny. U.S. Pat. No. 5,750,376, May 12, 1998.

Weiss, et al., In vitro growth and proliferation of multipotent neural stem cells and their progeny. U.S. Pat. No. 5,851,832 [Dec. 22, 1998].

Other References

Azizi, S. A., et al., “Engraftment and migration of human bone marrow stromal cells implanted in the brains of albino rats-similarities to astrocyte grafts,” Proc. Natl, Acad. Sci. USA, 95:3908-3913 [1998].

Bastians, et al., “Cell cycle-regulated proteolysis of mitotic proteins,” Mol. Biol. Cell, 10:3927-3941 [1999].

Bjornson, C. R. R., et al., “Turning brain into blood: a hematopoietic fate adopted by neural stem cells in vivo,” Science 283:534-537 [1999].

Celis, J. E., ed., Cell Biology: A Laboratory Handbook, 2nd edition, Academic Press, San Diego [1998].

Clarke, D. L., et al., “Generalized potential of adult neural stem cells,” Science 288:1660-1663 (2000).

Cong, Y. S. and S. Bacchetti, “Histone deacetylation is involved in the transcriptional repression of hTERT in normal human cells,” J. Biol. Chem., 275:35665-35668 [2000].

Dieffenbach and Dveksler, ed., PCR Primer: A Laboratory Manual, Cold Spring Harbor Laboratory Press, 1995.

Ferguson, et al., “High frequency of hypermethylation at the 14-3-3 s locus leads to gene silencing in breast cancer,” Proc. Natl. Acad. Sci. USA, 97:6049-6054 [2000].

Ferrel, J. E., “Xenopus oocyte maturation: new lessons from a good egg,” BioEssays, 21:833-842 (1999).

Fry, C. J., and C. L. Peterson, “Chromatin remodeling enzymes: who's on first?” Current Biology, 11:R185-R197 (2001).

Gage, F. H., “Mammalian neural stem cells,” Science 287:1433-1438 (2000).

Hansis, C, et al., “Oct-4 expression in inner cell mass and trophectoderm of human blastocysts,” Mol. Hum. Reprod. 6:999-1004 (2000).

Harlow and Lane, Antibodies: A Laboratory Manual, Cold Spring Harbor Laboratory Press, 1988.

Hatada, S., et al., “Gene correction in hematopoietic progenitor cells by homologous recombination,” Proc. Natl. Acad. Sci. USA, 97:13807-13811 [2000].

Herman et al., “Methylation-specific PCR: a novel PCR assay for methylation status of CpG islands,” Proc. Natl. Acad. Sci. USA, 93:9821-9826 [1996].

Hokland, et al., “Isolation of mononuclear cells from human blood and bone marrow and identification of leukocyte subsets by multiparameter flow cytometry,” Cell Biology: A Laboratory Handbook, 2nd edition, J. E. Celis, ed., Academic Press, San Diego [1998].

Jackson, K. A., et al., “Hematopoietic potential of stem cells isolated from murine skeletal muscle,” Proc. Natl. Acad. Sci. USA 96:14482-14486 [1999].

Jones, G. E., ed., Human Cell Culture Protocols in Methods in Molecular Medicine, Humana Press Inc., Totowa, N.J., 1996.

Keisari, Y., “Human Mononuclear Phagocytes in Tissue Culture,” in Methods in Molecular Medicine: Human Cell Culture Protocols (G. E. Jones, ed.), Humana Press Inc., Totowa, N.J. (1996).

Kominato, Y., Y. Hata, H. Takizawa, T. Tsuchiya, J. Tsukada, and F. Yamamoto, “Expression of human histo-blood group ABO genes is dependent upon DNA methylation of the promoter region,” J. Biol. Chem. 274: 37240-37250 (1999).

Kopen, G. C., et al., “Marrow stromal cells migrate throughout forebrain and cerebellum, and they differentiate into astrocytes after injection into neonatal mouse brains,” Proc. Natl, Acad. Sci. USA, 96:10711-10716 [1999].

Lanza R P, Cibelli J B, Blackwell C, Cristofalo V J, Francis M K, Baerlocher G M, Mak J, Schertzer M, Chavez E A, Sawyer N, Lansdorp P M, West M D, “Extension of cell life-span and telomere length in animals cloned from senescent somatic cells,” Science 288:665-9 (2000).

Lee et al., “Efficient generation of midbrain and hindbrain neurons from mouse embryonic stem cells,” Nature Biotechnology, 18:675-679 (2000).

Lemischka, I., “The power of stem cells reconsidered?” Proc. Natl. Acad. Sci. USA 96:14193-14195 [1999].

Lewin, M., “Tat peptide-derived magnetic nanoparticles allow in vivo tracking and recovery of progenitor cells,” Nat. Biotech., 18:410-414 [2000].

Limat, Alain and Thomas Hunziker, “Cultivation of Keratinocytes from the Outer Root Sheath of Human Hair,” in Methods in Molecular Medicine: Human Cell Culture Protocols (G. E. Jones, ed.), Humana Press Inc., Totowa, N.J. (1996).

Lu, Z. H., et al., “DNA replication in quiescent cell nuclei: regulation by the nuclear envelope and chromatin structure,” Mol. Biol. Cell, 10:4091-4106 (1999).

Lumelsky, et al., “Differentiation of embryonic stem cells to insulin-secreting structures similar to pancreatic islets,” Science, 292:1389-1394 (2001).

Mansouri, Ahmed, “Gene Targeting by Homologous Recombination in Embryonic Stem Cells,” in Cell Biology: A Laboratory Handbook, 2^{nd} edition, vol. 3 (J. E. Celis, ed.), Academic Press, San Diego (1998).

Munsie et al., “Isolation of pluripotent embryonic stem cells from reprogrammed adult mouse somatic cell nuclei,” Curr. Biol., 10:989-992 (2000).

Nichols, J., B. Zevnik, K. Anastassiadis, H. Niwa, D. Klewe-Nebenius, I. Chambers, H. Schöler, and A. Smith, “Formation of pluripotent stem cells in the mammalian embryo depends on the POU transcription factor Oct-4,” Cell 95: 379-391 (1998).

Pagano, et al., “Isolation and characterization of neural stem cells from the adult human olfactory bulb,” Stem Cells 18:295-300[2000].

Pittenger, M. F., et al., “Multilineage potential of adult human mesenchymal stem cells,” Science, 284:143-147 [1999].

Qui, L., A. Burgess, D. P Fairlie, H. Leonard, P. G. Parsons, B. G. Gabrielli, “Histone deacetylase inhibitors trigger a G2 checkpoint in normal cells that is defective in tumor cells,” Mol. Biol. Cell 11: 2069-2083 (2000).

Reubinoff, B. E., M. F. Pera, and Chui-Yee, “Embryonic stem cell lines from human blastocysts: somatic differentiation in vitro,” Nat. Biotech. 18: 399-404 (2000).

Sambrook, Fritsch, and Maniatis, Molecular Cloning: A Laboratory Manual, Cold Spring Harbor Laboratory Press, 1989.

Schuldiner et al., “Effects of eight growth factors on the differentiation of cells derived from human embryonic stem cells,” Proc. Natl. Acad. Sci. USA 97: 11307-11312 [2000].

Shamblott, M. J., et al., “Derivation of pluripotent stem cells from cultured human primordial germ cells,” Proc. Natl. Acad. Sci. USA 95: 13726-13731 (1998).

Sly, W. S. and J. Grubb, “Isolation of Fibroblasts from Patients,” in Methods in Enzymology, vol. 58 (W. B. Jakoby and I. H. Pastan, eds.), Academic Press, London (1979).

Tada, M., T. Tada, L. Lefebvre, S. C. Barton and M. A. Surani, “Embryonic germ cells induce epigenetic reprogramming of somatic nucleus in hybrid cells,” EMBO J. 16: 6510-6520 (1997).

Thomson, J. A., et al., “Embryonic stem cell lines derived from human blastocysts,” Science 282: 1145-1147 (1998).

Wakayama, et al., “Full-term development of mice from enucleated oocytes injected with cumulus cell nuclei,” Nature, 394:369-374 [1998].

Walsh, C. P. and T. H. Bestor, “Cytosine methylation and mammalian development,” Genes & Dev., 13:26-34 [1999].

Wang, X. M., et al., “Mos oncogene product associates with kinetochores in mammalian somatic cells and disrupts mitotic progression,” Proc. Natl. Acad. Sci. USA, 91:8329-8333 [1994].

Wilmut, I., et al., “Viable offspring derived from fetal and adult mammalian cells,” Nature, 385:810-813 [1997].

Yamamoto, “Expression of human histo-blood group ABO genes is dependent upon DNA methylation of the promoter region,” J. Biol. Chem. 274: 37240-37250 (1999).

Zuk P A, Zhu M, Mizuno H, Huang J, Futrell J W, Katz A J, Benhaim P, Lorenz H P, Hedrick M H, “Multilineage cells from human adipose tissue: implications for cell-based therapies,” Tissue Eng. 7:211-28 (2001).

### SUMMARY OF THE INVENTION

An objective of the present invention is to provide a method to produce mammalian, preferably human, pluripotent stem cell lines from any adult somatic cell without using fetal or embryonic tissue. Adult somatic cells are treated to reverse the epigenetic changes that occur during differentiation, resulting in cells that are pluripotent. The resultant pluripotent cells are referred to herein as in vitro derived adult pluripotent stem cells or NucRem™ cells. Examples of adult somatic cells which may be used as the starting material for the in vitro derived adult pluripotent stem cells or NucRem™ cells, include dermal fibroblasts, epidermal cells, keratinocytes, hair outer root sheath cells, and peripheral blood monocytes.

Adult somatic cells are obtained from animals, preferably human subjects, and cultured according to standard cell culture protocols available to those of ordinary skill in the art, for example as described in Methods in Molecular Medicine: Human Cell Culture Protocols (G. E. Jones, ed.), Humana Press Inc., Totowa, N.J. (1996). Expanded cultures of adult somatic cells are subsequently treated to remove or reverse the affects of tissue specific epigenetic changes in chromosome architecture and patterns of gene expression. Examples of epigenetic changes include DNA methylation, bound transcription activators or repressors, and bound histone deacetylase (HDAC) or deacetylated histones. The resulting reprogrammed cells may resemble embryonic stem cells in patterns of gene expression and/or pluripotency. These cells can be continuously passaged and survive cryopreservation.

Another object of the invention is to produce tissue specific autologous (self) progenitor cells derived from said in vitro derived adult pluripotent stem cells or NucRem™ cells. These progenitor cells may be used in cell therapy applications to treat diseases of cellular degeneration. Diseases of cellular degeneration include for example neurodegenerative diseases such as stroke, Alzheimer's disease Parkinson's disease, multiple sclerosis, Amyotrophic lateral sclerosis, macular degeneration, osteolytic diseases such as osteoporosis, osteoarthritis, bone fractures, bone breaks, diabetes and liver injury and degenerative diseases, myocardial infarct, burns and cancer. It is envisioned that in vitro derived adult pluripotent stem cells or NucRem™ cells, progenitor cells or fully differentiated cells derived from these cells may be implanted or transplanted into a host. An advantage of the invention is that large numbers of autologous stem cells can be produced for implantation without the risk of immune system mediated rejection.

Another object of the invention is to provide a method to produce ex vivo engineered tissues for subsequent implantation or transplantation into a host, wherein the cellular components of said engineered tissues are the in vitro derived adult pluripotent stem cells or NucRem™ cells of the present inventions or cells derived therefrom.

Another object of the invention is a method of producing a useful pharmaceutical product, wherein the in vitro derived adult pluripotent stem cells or NucRem™ cells or cells derived therefrom may be transformed with a gene-of-interest, which encodes a useful gene product. It is envisioned that said transformed cells may be grown in vitro in a bioreactor to produce the useful gene product. Alternatively, the transformed cells may be implanted into a host, preferably a human suffering from a disease of genetic deficiency.

Another object of the invention is the provision of a method to screen for prospective drugs or agents which mediate the differentiation of cells. It is further envisioned that the in vitro derived adult pluripotent stem cells or NucRem™ cells of the present invention may be used in lieu of human embryonic stem cells as a model cell line to study the differentiation of human cells.

An important advantage of this invention is that the cellular reprogramming procedure does not involve the use of human embryonic stem cells, human embryonic carcinoma cells, or human primordial germ cells. Another advantage is that the reprogramming procedure does not involve the creation of human embryos or human/animal chimeras through nuclear transfer, or fusion of somatic cells with oocytes.

### BRIEF DESCRIPTION OF DRAWINGS

FIG. 1 depicts photomicrographs of cultures of human adult keratinocytes before treatment (panel A) and cultures of in vitro derived adult pluripotent stem cells treated with retinoic acid for three (3) days (panels B-D).

FIG. 2 is a histogram depicting the percent relative change in the expression of several marker genes after various cell culture treatment regimens compared to human adult keratinocytes. Treatment regimen 1 consists of 5-aza-2′-deoxycytidine and trichostatin A treatment. Treatment regimen 2 consists of 5-aza-2′-deoxycytidine, trichostatin A and Tat-cyclin B treatment. Treatment regimen 3 consists of 5-aza-2′-deoxycytidine, trichostatin A, Tat-cyclin B and retinoic acid treatment. hTRT connotes telomerase, NF connotes neurofilament, alpha AT connotes α1-antitrypsin and cACT connotes cardiac actin.

### DETAILED DESCRIPTION OF THE INVENTION

Introduction

Particular embodiments of the present invention described herein are not intended to limit the scope of the present invention. Although any methods, compositions, reagents, cells, similar or equivalent to those described herein may be used in the practice or testing of the invention, preferred methods and materials are described herein. Throughout this description, the embodiments of the invention should be considered as exemplars, rather than as limitations on the present invention.

The present invention is directed to a method of producing pluripotent adult stem cells from non-embryonic somatic cells. The method comprises establishing a culture of the cells and treating the cells to reverse specific epigenetic chromosomal changes associated with differentiation. Throughout the described invention, unless otherwise indicated, all cells are cultured as adherent cells on tissue culture dishes or flasks at 37° centigrade in an atmosphere containing 5-10% CO₂.

As used herein, the term “germ layers” means the primordial embryonic tissues including ectoderm, which gives rise to, for example, the integument and nervous system; mesoderm, which gives rise to, for example, bone, muscle, and blood: and endoderm, which gives rise to, for example, the lining of the gut, liver, and kidney.

As used herein, “pluripotency”, “pluripotent” or “pluripotential” means the potential capacity of a cell or cells to give rise to any one of multiple different cell types derived from any of the three germ layers. Such cells include, but are not limited to neurons, epidermal cells, osteoblasts, osteocytes, hepatocytes, blood cells, cardiomyocytes, myocytes or progenitors thereof. One skilled in the art will readily appreciate that pluripotent stem cells can give rise to many different tissues or cell types in the body.

As used herein, the term “stem cell” means a cell that is capable of self-renewal. Stem cells may be immortal, i.e., capable of unlimited proliferation, or they may have a limited capacity for proliferation. However, stem cells must be able to divide at least once in culture.

As used herein, “adult somatic cell” means a diploid cell that is not a germ cell or germ cell. However, said adult somatic cell may be a diploid germ cell precursor. Adult somatic cells are obtained from a non-embryo, non-fetus individual. Preferably the non-embryo individual is a human. Examples of adult somatic cells include, for example, epithelial cells, neurons, glial cells, epidermal cells, keratinocytes, chondrocytes, lymphocytes and the like.

As used herein, the term “in vitro derived adult pluripotent stem cell” or “NUCREM™ cell” means a pluripotent cell with some capacity for self-renewal, which is cultured or derived from any adult somatic cell without the step of cell fusion or nuclear transfer. In a preferred embodiment, adult somatic cells are treated in vitro to remodel the chromatin to allow for the expression of gene products associated with stem cell activity and pluripotentiality. The term “remodeled cell” may be used interchangeably with in vitro derived adult pluripotent stem cell” or “NUCREM™ cell”.

As used herein, “chromatin remodeling”, “remodel chromatin”, “nuclear remodeling”, “remodel nuclei” “nuclear reprogramming” or “cellular reprogramming” means any degree of alteration or erasure of heritable patterns of nucleic acid methylation, chromatin condensation, epigenetic changes, genomic imprinting or the like, which occurs as a result of the differentiation of cells during development of a fertilized egg into an adult. As used herein, the term “adult” means any non-embryo, including new born infants.

As used herein, “normal growth medium” is defined as the medium and/or growth conditions used to culture a particular adult primary cell line. For example, normal growth medium for hair outer root sheath cells (“ORS”) cells is keratinocyte growth medium (KGM), which is commercially available from Clonetics Corporation (San Diego, Calif.). Normal growth media for particular cell types are generally known by those skilled in the art.

As used herein, “agent” means any drug, reagent, ion, compound, nucleic acid or peptide that affects a change in the pattern of gene expression, chromatin structure or cell morphology. Agent includes “morphogenic growth factors”, which is defined below.

As used herein, the term “differentiate”, “differentiation”, “differentiated” or “differentiating” means any change in cellular gene expression accompanied by or accompanying the restriction of a cell and its progeny to a more specific cell-type lineage. For example, changes in gene expression accompany the differentiation of a glial cell from a neural stem cell precursor or the differentiation of an osteoclast from a bone marrow stem cell.

In vitro Remodeling of Adult Somatic Cells to Produce Pluripotent Stem Cells

Reversal of Epigenetic Changes which Occur During Cell Differentiation

Heritable changes in gene expression that occur during cell differentiation are due in part to epigenetic changes in chromosomal conformation. It is well known in the art that loosely condensed regions of chromosomes contain transcriptionally active genes and highly condensed regions of chromosomes contain transcriptionally silenced genes. The state of chromosome condensation and transcription activity is controlled in part by DNA methylation and histone acetylation (reviewed in Walsh and Bestor, 1999, Kominato et al., 1999, Cong and Bacchetti, 2000). Methylation or hypermethylation of cytosines within CpG promoters is associated with gene silencing (Ferguson et al., 2000), whereas unmethylated DNA is generally transcriptionally active (Kominato et al., 1999).

Differentiated adult somatic cells show stable and specific patterns of methylation, whereas pluripotent cells, such as primordial germ cells and preimplantation embryos, show genome-wide patterns of demethylation (reviewed in Tada et al., 1997). A few studies have demonstrated that these heritable patterns of methylation can be reversed. For example, Tada et al. (1997) fused murine thymic lymphocytes with murine embryonic germ cells and demonstrated the genome-wide demethylation of the lymphocyte cell nucleus. The resulting demethylated nucleus was subsequently shown to be pluripotent.

The present invention is directed to a method of reprogramming adult somatic cells, wherein the method comprises the step of DNA demethylation. As used herein, the term “demethylation” means the removal of methyl groups from nucleotides comprising DNA. Demethylation also means the inhibition of methylation of nucleotides comprising DNA. According to the present invention, adult somatic cells may be treated with an agent to promote or induce the demethylation of DNA. In one embodiment of the demethylation step, adult somatic cells are treated with 5-aza-2′-deoxycytidine (see Kominato et al., 1999, which is incorporated herein by reference). Primary adult somatic cells are cultured in normal growth medium in the presence of 0.1 to 100 μM of 5-aza-2′-deoxycytidine (Sigma Chemical Co., St. Louis, Mo.), for 1 to 10 days, preferably 5 days, to promote or induce demethylation of DNA. It is envisioned that other reagents may be used in the demethylation step, including, for example, methylase specific antibodies or other inhibitors of methylases.

In addition to specific patterns of DNA methylation and demethylation, global patterns of transcription are also regulated by chromatin remodeling enzymes, such as histone acetylases and deacetylases. Acetylated histones bind to DNA with lower affinity than deacetylated histones, thereby generally permitting transcription factors to bind to DNA. Conversely, deacetylated histones bind DNA with higher affinity, blocking the access of transcription activators to DNA, thereby generally repressing transcription. In another embodiment of the invention, primary adult somatic cells are reprogrammed via inhibition of or reversal of histone deacetylation. Primary adult somatic cells are cultured in normal growth medium in the presence of 0.1-10,000 ng/ml of trichostatin A (Sigma Chemical Co., St. Louis, Mo.) for at least 24 hours. Trichostatin A treatment of cells has been shown to induce or allow the expression of previously silenced genes (Qui et al., 2000 and Cong and Bacchetti, 2000). Alternatively, cells may be treated with sodium butyrate, which also inhibits histone deacetylation. It is envisioned that any reagent which induces or facilitates changes in histone acetylation or DNA methylation may be used in the practice of this invention.

In yet another embodiment, primary adult somatic cells are treated with a chromatin remodeling protein preferably nucleoplasmin, which is a nuclear chaperone that facilitates the exchange of histone H1 with histone B4 and HMG1, thereby facilitating activation of transcription (Lu et al., 1999). In a preferred embodiment, a transit peptide (e.g., Tat) is fused to a peptide comprising nucleoplasmin which is administered to cells in normal medium. Histone exchange is allowed to proceed before the nucleoplasmin treatment is stopped. It is envisioned that cells may be treated with any chromatin remodeling enzyme, reagent, intercalating agent, or combination thereof, that is known in the art, which facilitates the removal of transcription repressors and nuclear remodeling. For a recent review on chromatin remodeling enzymes, see Fry and Peterson, 2001, which is incorporated herein by reference.

As used herein, the term “nuclear chaperone” means any reagent that facilitates the exchange of histone H1 or other transcription repressors for HMG1, histone B4 or other transcription activators.

In another embodiment, primary adult somatic cells are treated with a combination of demethylation agents, deacetylation inhibitors or acetylation promoters and/or nuclear chaperones to promote nuclear reprogramming. Additionally, the skilled artisan may treat the primary cells with other reagents known in the art to block DNA methylation, promote DNA demethylation, block histone deacetylation, promote histone acetylation, and/or promote the exchange of histone H1 with histone B4 or HMG1, in order to reprogram the genome of said cells.

Activation of Reprogrammed Cells

It is well known in the art that somatic cell nuclei (differentiated) which are transferred into enucleated mature oocytes can be remodeled (dedifferentiated or reprogrammed), thus permitting the production of complete embryos (Wilmut et al., 1997, Wakayama, et al., 1998, Stice, 1999, 2001). In the present invention, adult somatic cells are cultured in an environment that is envisioned to mimic in part the molecular environment of mature oocytes. It is well known in the art that mature mammalian oocytes are arrested in metaphase of meiosis II until activated by sperm. Cell cycle arrest in metaphase II is maintained in mature oocytes by the prevention of proteosome-mediated degradation of G2-M cyclins, particularly cyclin-B, through the activity of the cytostatic factor c-Mos (reviewed in Ferrel, 1999, Bastians et al.,1999, Wang et al., 1994).

In the present invention, cultures of adult somatic cells are treated with either one or more of the following reagents to induce metaphase arrest: G2-M cyclins, for example cyclin-A or cyclin-B, c-Mos, colchicine, colcemid or any other reversible microtubule drug. Polypeptide reagents, such as cyclin-A, cyclin-B or c-Mos, are administered to cells through membrane translocation methods including, but not limited to, microinjection, liposome-mediated translocation, or direct translocation of polypeptides which are fused to transit peptides. Alternatively, vectors comprised of polynucleotides encoding cyclin-A, cyclin-B or c-Mos, for example, under the control of a regulated promoter, such as the commercially available Tet-on/Tet-off system (Clontech, Palo Alto Calif.), are transfected into cultured cells via cationic lipid transduction, microinjection, or electroporation. After metaphase arrest is sustained in the cell for at least 1 to 6 hours, the cell is released from metaphase arrest by media replacement, as in the case of treatment by peptide or microtubule poison, or by promoter repression, as in the case of polynucleotide vector transfection.

Preferred Method of Somatic Cell Remodeling

In the preferred embodiment of this invention, easily obtainable adult somatic cells, most preferably hair outer root sheath (ORS) cells, epidermal keratinocytes or buccal epithelial cells are obtained from a subject and expanded in culture, as described herein, wherein the subject is preferably a human. The cells are treated with an amount of a demethylation agent, preferably about 10 μM 5-aza-2′-deoxycytidine for about 5 days, to induce global genomic demethylation. These cells may also be treated with a deacetylation inhibitor or acetylation promoter, preferably 100 ng/ml or 1 μM of trichostatin A for about 24 hours, to promote histone acetylation. These cells may also be treated with an amount of a polypeptide comprising a nuclear chaperone or other chromatin remodeling enzyme (Fry and Peterson, supra), preferably nucleoplasmin or tat-nucleoplasmin, to facilitate the removal of transcription repressors from the DNA.

The nucleoplasmin used in an embodiment of the invention may be produced according to the following protocol, which is provided as an example only and is not to be construed as limiting. A recombinant polynucleotide encoding a chimeric Tat-nucleoplasmin fusion polypeptide is produced by the polymerase chain reaction using an upstream oligonucleotide primer encoding the Tat peptide and the first 20 nucleotides of the nucleoplasmin open reading frame. The nucleoplasmin sequence may be obtained from the public database (e.g., GenBank Accession No. AF081280) and the full-length nucleoplasmin cDNA may be obtained from the IMAGE consortium (IMAGE Clone No. 4182870). The Tat-nucleoplasmin polynucleotide is cloned into the EcoR1 and Not1 sites of the pGAPZαA Pichia expression vector (Invitrogen, Carlsbad, Calif.), and transformed into the yeast Hansenula polymorpha according to the instructions provided by the vendor. The transformed Hansenula is grown under standard yeast culture conditions to an OD₆₀₀ of 0.6 to 1.2. The culture supernatant is concentrated using CENTRICON PLUS YM-10® concentrators (Millipore, Bedford, Mass.) to obtain a Tat-nucleoplasmin peptide concentration of 1,000 μg/ml.

Subsequent to the aforementioned step or steps, the cells are then treated with an amount of an agent that arrests cells in metaphase, preferably a polypeptide comprising cyclin-A or cyclin-B, for 30 hours to induce prolonged mitotic arrest. The cells are then released from the mitotic arrest by washing the cells in at least one change of culture medium.

The cyclin-A or cyclin-B used in the preferred embodiment of the invention may be produced according to the following protocol. A recombinant polynucleotide encoding a chimeric Tat-cyclin A or B fusion polypeptide is produced by the polymerase chain reaction using an upstream oligonucleotide primer encoding the Tat peptide and the first 20 nucleotides of the cyclin A or B open reading frame. The Tat peptide sequence, and derivations thereof, are well known in the art and are provided in the literature (for example, see Lewin et al, 2000). For example, the cyclin B sequence may be obtained from the public database (GenBank Accession No. X58708) and a full length cyclin B cDNA may be obtained from the IMAGE consortium (IMAGE Clone No. 1499287). The Tat-cyclin A or B polynucleotide may be cloned into the EcoR1 and Not1 sites of the pGAPZαA Pichia expression vector (Invitrogen, Carlsbad, Calif.), and transformed into the yeast Hansenula polymorpha according to the instructions provided by the vendor. The transformed Hansenula is grown under standard yeast culture conditions to an OD₆₀₀ of 0.6 to 1.2. The culture supernatant is concentrated using CENTRICON PLUS YM-10® concentrators (Millipore, Bedford, Mass.) to obtain a Tat-cyclin B peptide concentration of 1,000 μg/ml. The preceding protocol is provided as an example only and is not to be construed as limiting. The skilled artisan may use any method known in the art to produce a cyclin polypeptide, including peptide synthesis or other recombinant DNA methodologies (see Sambrook, et al., “Molecular Cloning”, 1989, which is incorporated herein by reference).

Subsequent to the mitotic arrest step, adherent cells are trypsinized, replated and cultured in media designed to support growth of stem cells. In a preferred embodiment, the remodeled cells are passaged onto a layer of mouse embryo fibroblast feeder cells in 80% KNOCKOUT® DMEM, 20% KNOCKOUT® SR (GIBCO/BRL, Bethesda Md.), 1 mM glutamine, 0.1 mM β-mercaptoethanol, 1% nonessential amino acid stock (GIBCO/BRL, Bethesda Md.), 4 ng/ml basic fibroblast growth factor, and 1,000 U/ml leukemia inhibitory factor (ES cell medium; Schuldiner et al., 2000). The KNOCKOUT® DMEM and KNOCKOUT® SR are special formulations designed to enhance the growth and maintain the pluripotentiality of embryonic stem cells. The skilled artisan may also use other cell media formulations, which are known in the art, to propagate pluripotent cells.

In another embodiment, remodeled cells are directly cultured under conditions that are not optimal for maintaining stem cells, but rather allow the remodeled cells to differentiate. Generally, such culture conditions may lack serum, lack feeder cells, contain a high density of cells, or contain one or more of various morphogenic growth or differentiation factors, such as retinoic acid or nerve growth factor.

Primary Cultures of Adult Somatic Cells

In the present invention, adult somatic cells, which include for example hair outer root sheath (“ORS”) cells, epidermal keratinocytes, circulating monocytes, fetal cord blood cells, dermal fibroblasts, peritoneal macrophages, squamous epithelial cells, or any other type of cell excluding germ cells, are isolated from an animal, most preferably a human. Primary cell cultures are then established using standard procedures that are well known in the art (Cell Biology: A Laboratory Handbook, 2^{nd} edition, [J. E. Celis, ed.], Academic Press, San Diego [1998], Methods in Molecular Medicine: Human Cell Culture Protocols [G. E. Jones, ed.], Humana Press Inc., Totowa, N.J. [1996], and references included therein, which are incorporated herein by reference). In a preferred embodiment, ORS cells are obtained from anagen hair follicles that are plucked from one of several anatomical sites and cultured according to standard protocols. The preferred method for obtaining and culturing ORS cells is described in detail in Limat and Hunziker (1996), which is herein incorporated by reference.

In another preferred embodiment, monocytes from humans or other mammals are obtained from blood samples using standard Ficoll-Paque density gradient centrifugation methods (Hokland, et al., 1998). Mononuclear phagocytes are then cultured according to standard protocols available in the art, such as the protocol described in Keisari (1996), which is incorporated herein by reference.

Other primary adult somatic cells, which are obtained from human or other mammalian sources and cultured according to standard protocols, may be used as starting material in this invention. Growth and culture of in vitro-derived adult pluripotent stem cells

Cultures of the in vitro derived adult pluripotent stem cells (“NUCREM™” cells) are propagated in media required to sustain growth and inhibit differentiation. Such media may contain exogenous factors, such as leukemia inhibitory factor (LIF), basic fibroblast growth factor (bFGF), or other factors. The preferred medium for propagation of the herein described in vitro-derived pluripotent stem cells (“NUCREM™ cells”) is ES cell medium, as described above and in Schuldiner et al. (2000).

Means of Assessing the Embryonic Stem Cell-Like Phenotype and Pluripotency

The resulting in vitro derived adult pluripotent stem cells may resemble embryonic stem cells in morphology and in biochemical histotype. In vitro derived adult pluripotent stem cells may be passaged several times in culture, maintained for several months in culture and/or survive cryopreservation.

The embryonic stem cell-like phenotype of the in vitro derived adult pluripotent stem cells may be determined as commonly described in the art (Thomson et al, 1998, Shamblott et al., 1998 and Reubinoff et al., 2000, which are herein incorporated by reference). It is envisioned that the in vitro derived adult pluripotent stem cells may express several molecular markers that are also expressed by EG or ES cells. For example, in vitro derived adult pluripotent stem cells may express the embryo-specific POU transcription factor Oct-4. Additionally, in vitro derived adult pluripotent stem cells may express the human telomerase (“hTRT”) gene product, which is indicative of cellular immortality. Oct-4 and or hTRT gene expression may be determined by one of many art recognized methods, such as reverse transcription-polymerase chain reaction (“RT-PCR”).

It is generally accepted in the art that pluripotent stem cells may be cultured many times while maintaining an undifferentiated state and while retaining the capacity to differentiate into a variety of cell and tissue types (Thomson et al., 1998 and Schuldiner et al., 2000). The ability of the in vitro derived adult pluripotent stem cells to differentiate into a wide variety of differentiated cell types can easily be tested by means commonly available to the skilled artisan. For example, to induce differentiation, in vitro derived adult pluripotent stem cells may be cultured for several weeks without passage onto fresh mouse fibroblast feeder layers or in the absence of LIF. Such suboptimal culture conditions induce the formation of embryoid bodies (EB), structures that appear to mimic early developmental processes and cell-to-cell interactions.

Embryoid bodies are fixed and sectioned according to methods commonly known in the art. The EB sections are examined for the presence of differentiated cell types using histological and molecular methods well known to the skilled artisan (as described in Shamblott, 1998, which is incorporated herein by reference). For example, the following proteins may be detected using commercially available antibodies: muscle-specific actin, which indicates myocytes; desmin, which indicates mesenchymal cells; CD34, which indicates vascular endothelium; neurofilament, which indicates neuronal cells; cytokeratin, which indicates epithelial cells; and alpha fetoprotein, which indicates endodermal derivatives. Furthermore, the mRNAs encoding the above described proteins may be detected using RT-PCR or other methods of detection commonly known in the art.

In vitro derived adult pluripotent stem cells may also be injected into severe combined immunodeficient (SCID) mice to induce formation of teratomas, which comprise many different cell types and indicate pluripotency (Thomson, 1998). Additionally, in vitro derived adult pluripotent stem cells may be induced to form lineage restricted cells via in vitro morphogenic growth factor treatment, as described in Schuldiner, et al. (2000), which is herein incorporated by reference. As used herein, the term “morphogenic growth factor” means any ion, molecular compound, cellular event or condition that stimulates or induces the differentiation of any cell. Morphogenic growth factors include, for example, cellular starvation, low pressure, high pressure, stretching of cells, bending of cells, change in temperature, change in pH, polypeptides, glycolipids, glycoproteins, components of the glycocalyx, components of the extracelluar matrix, steroids, lipid soluble compounds such as retinoic acid, amino acids and calcium. The presence of specific lineage restricted (differentiated or differentiating) cell types is assessed by histological and molecular methods well known to the skilled artisan, as described herein.

Therapeutic Applications

In another embodiment, the in vitro-derived adult pluripotent stem cells (“NUCREM™ cells”) described herein can be used in any research or medical application where human embryonic stem cells, human adult stem cells, or any other pluripotent, multipotent, or lineage precursor cells are used. In one embodiment, the present invention may be used in cell-based assays to identify useful pharmaceuticals and medicaments. The in vitro derived adult pluripotent stem cells may be treated with an agent, drug, virus, polypeptide or other ion or compound and assessed for the expression of cell differentiation markers.

In another embodiment, the present invention provides for the production of differentiated cells for replacement or repair of damaged or impaired cells or tissues in the treatment of degenerative diseases or injuries such as, for example neurodegenerative diseases such as stroke, Alzheimer's disease Parkinson's disease, multiple sclerosis, Amyotrophic lateral sclerosis, macular degeneration, osteolytic diseases such as osteoporosis, osteoarthritis, bone fractures, bone breaks, diabetes and liver injury and degenerative diseases, myocardial infarct, burns and cancer. The present invention may also be used as replacement cells to treat injuries which require tissue grafts, such as bone marrow transplant, bone grafting, cartilage repair, skin grafts, and spinal cord injury, for example.

In vitro derived adult pluripotent stem cells may be expanded in culture to produce large quantities of cells, i.e., on the order of 10⁶ to 10⁹ cells, to be implanted or injected into a subject. In one embodiment, in vitro derived adult pluripotent stem cells or in vitro derived adult pluripotent stem cells that have been differentiated to varying degrees may be injected directly into damaged tissue, such as damaged heart muscle, brain tissue, bone or joints. Said cells may be non-encapsulated or encapsulated within a biocompatible matrix or polymer. In another embodiment, in vitro derived adult pluripotent stem cells or in vitro derived adult pluripotent stem cells that have been subjected to varying degrees of differentiation may be seeded onto tissue engineered biomaterials or surface for the purpose of generating immunologically compatible organs or tissue replacements. As used herein the term “engineered surface” means any substance designed for the production of tissue engineered tissues or organs. Engineered surfaces may be natural or synthetic polymers. Engineered surfaces may by coated with biologically active molecules such as laminin or fibronectin to enhance cell growth or differentiation. For example, engineered surfaces include, but are not limited to collagen mesh, which may be used to make engineered arteries, and polylactate/glycolate polymers used to deliver growth factors or to encapsulate cells. Engineered surfaces may be formed into any shape to facilitate design of the tissue or organ.

Expanded cultures of the instant in vitro derived adult pluripotent stem cells may be differentiated by in vitro treatment with growth factors and/or morphogens (Schuldiner et al., 2000, and as described above). Populations of differentiated cells are then implanted into the recipient host near the site of injury or damage, or cultured in vitro to generate engineered tissues, as described. In a preferred embodiment to produce differentiated cells, approximately 10⁶ to 10⁸in vitro-derived adult stem cells (“NUCREM™ cells”) are trypsinized and passaged onto a 100 mm plastic petri dish. Cells are cultured for 5 days in the absence of a feeder layer, LIF, and/or bFGF to induce the formation of embryoid bodies. The embryoid bodies are collected and disassociated by treatment with trypsin then plated onto a 100 mm tissue-culture dish. The monolayer cultures thus established are then treated with various growth factors or morphogens to induce differentiation along one or more particular pathways.

Genetic Modification of Cells

In another embodiment, the in vitro derived adult pluripotent stem cells of the present invention may be genetically modified to express one or more specific genes-of-interest or to disrupt the expression of specific genes (Hatada et al., 2000). As used herein, the phase “genetically modified” means any modification or alteration in the sequence of any portion of the entire genomic sequence of a cell, including the mitochondrial as well as nuclear genome, and further including the addition of ectopic nucleic acids to the cell as in a plasmid or artificial chromosome or portion thereof. Exogenous DNA may be transferred to the cells by electroporation, calcium phosphate, microinjection, lipofection, retroviral or other viral or microbial vectors or other means commonly known in the art (Celis, J. E., 1998). Said genetically modified cells could be used in bioreactors to produce pharmaceutical products, or in cell therapy treatments for genetic diseases such as cancer, Cystic Fibrosis, adenosine deaminase deficiency (“ADA”), Osteogenesis imperfecta, Hemophilia, or Tay-Sachs disease, for example. In the treatment of a genetic disease, genetically modified cells of the present invention may be administered to the patient near the site of the defect.

All references cited herein are hereby incorporated by reference. The specification described above provides several preferred embodiments of the invention and is intended to enable the invention. Preferred embodiments are further described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLE 1

### Culturing Procedure for Human Outer Root Sheath (ORS) Cells

Hairs are plucked, using forceps, from the occipital region of the scalp. Follicles containing visible ORS tissues, as determined under the dissecting microscope, are snipped off with fine scissors and collected in a 60-mm dish containing 5 ml of solution A (DMEM buffered with 0.25 mM HEPES pH 7.2, 10% fetal calf serum (FCS), 40 U/ml penicillin, 40 μg/ml streptomycin). Follicles are rinsed at least four times in sterile solution A, transferred to a 35 mM petri plate, covered with solution B (trypsin [0.1%]/EDTA [0.02%]) solution, then incubated at 37° C. until the ORS cells completely detach (15 to 20 minutes). Five volumes of solution C (64% DMEM, 25% Ham's F-12, 1% adenine, 0.1% insulin, 0.1% triiodothryonine, 0.2% hydrocortisone, 1% glutamine, 0.01% epidermal growth factor (EGF), 0.1% choleratoxin, 1% penicillin/streptomycin, 1% fungizone, 10% FCS) are added to the follicles to stop the digestion. The cell suspension is passed through a 5 ml pipette several times to disperse the cells.

The ORS cells are plated onto a feeder layer of postmitotic dermal fibroblasts and cultured in the presence of solution C. Human dermal fibroblasts are available from the American Type Culture Collection and cultured according to the instructions provided. Human dermal fibroblasts are rendered postmitotic by treatment with 8 μg/ml mitomycin C for five hours.

The first medium change is done to the primary ORS cultures at day seven, and subsequent medium changes are done three times per week thereafter. When the culture is 80% to 100% confluent, the residual feeder cells are removed via EDTA (0.02%) treatment. The ORS cells are treated with 0.5 ml of solution B for approximately 10 minutes. 1.5 ml of solution C is added to the dish and the cells are suspended by vigorous pipeting. Cells are counted in a hemocytometer and then plated at a density of no less than 1000 cells per square centimeter to establish secondary cultures. Secondary cultures of ORS cells are cultured in Keratinocyte Growth Medium (“KGM”) (Clonetics Corporation, San Diego Calif.).

### EXAMPLE 2

### Production of In Vitro Derived Adult Pluripotent Stem Cells from Keratinocytes

Human adult keratinocytes were obtained from Clonetics (San Diego, Calif.) and grown in Keratinocyte Growth Medium in 5-10% CO₂ at 37° C., according to the instructions provided by the manufacturer (“Keratinocyte System Instructions”, BioWhittaker catalogue number AA-1000).

5-aza-2′-deoxycytidine (Sigma, St. Louis) was added at a concentration of 10-25 μM to cultures of human adult keratinocytes that were approximately 40% to 80% confluent. These cultures were further incubated for approximately four (4) days at 37° C. in an atmosphere of 5-10% CO₂. After four days in the presence of 5-aza-2′-deoxycytidine, trichostatin A (Sigma, St. Louis) was added to the cultures at a concentration of 100-250 ng/ml. Cultures were further incubated for approximately one (1) day, as described above. RNA was extracted from some of the aliquots of cultures at this point (treatment regimen 1) for subsequent RT-PCR analysis. Other aliquots of cultures were subsequently treated as follows.

After 5 days (cumulative) of 5-aza-2′ deoxycytidine treatment and 24 hours of trichostatin A treatment, the cell cultures were washed several times with KNOCKOUT® DMEM (GIBCO/BRL, Gaithersburg, Md.) media. After the final wash, cells were cultured for approximately 30 hours in KNOCKOUT® DMEM without serum, containing 10-10,000 ng/ml of Tat-Cyclin B (SEQ ID NO:1). After 30 hours of Tat-Cyclin B treatment (treatment regimen 2), RNA was extracted from some aliquots of cells for subsequent RT-PCR analysis and other aliquots of cultures were treated with retinoic acid, as described below.

### EXAMPLE 3

### Retinoic Acid Induced Differentiation of In Vitro Derived Adult Pluripotent Stem Cells

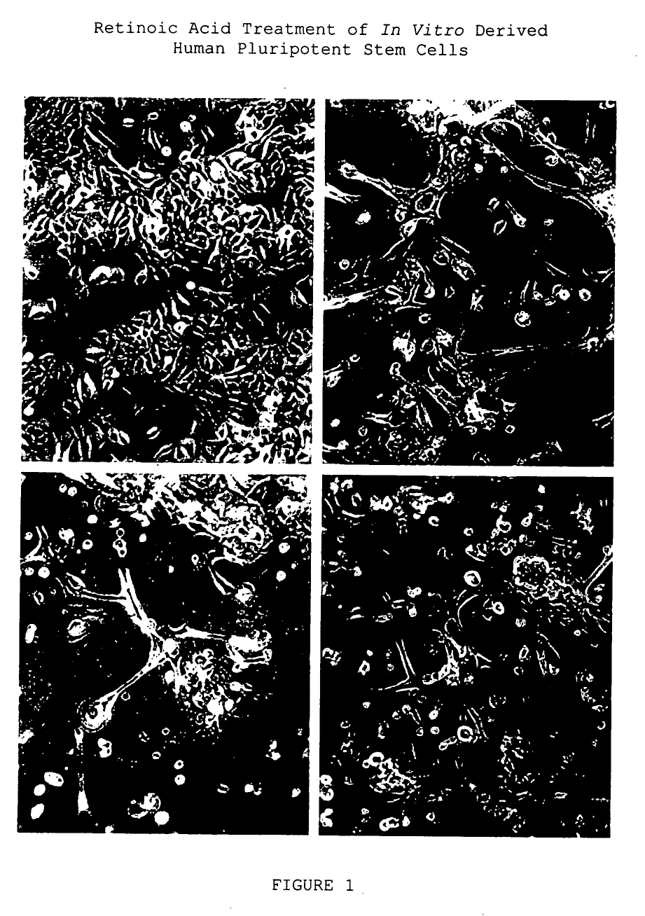

Subsequent to cyclin B treatment, the cultures of in vitro derived adult pluripotent stem cells were cultured in the presence of approximately 1-2 μM retinoic acid (“RA”) (Sigma, St. Louis) for five (5) to 10 days. After three (3) days, cells began to sprout extensions that closely resembled neurites (FIG. 1; compare panel A, which depicts untreated human adult keratinocytes, to panels B-D, which depict in vitro derived adult pluripotent stem cells [“NUCREM™ cells”] after 3 days of RA treatment). By day five (5) of retinoic acid treatment, approximately 50% of the cells in culture attained a neuron-like morphology (FIG. 1). At day five of RA treatment (treatment regimen 3), RNA was extracted from the cultures for RT-PCR analysis.

### EXAMPLE 4

### RNA Extraction and RT-PCR

To monitor the differential expression of various genes in the in vitro-derived adult pluripotent stem cells (“NUCREM™ cells”) and RA induced differentiated cells that were derived from said NUCREM™ cells, reverse transcription-polymerase chain reaction (RT-PCR) was performed.

It is generally accepted in the art that stem cell populations in the body, whose purpose comprises, among other functions, the repair and regeneration of tissues, must be able to continually divide throughout the life of the organism. Such cells must be able to regenerate their telomeres, and hence such cells express the telomere regeneration enzyme telomerase (“TRT”). Therefore, TRT RNA levels in cells obtained from various steps along the remodeling process were measured by semi-quantitative RT-PCR. It is envisioned that prior to being remodeled, keratinocytes will express low levels of TRT, whereas after remodeling, the in vitro-derived adult pluripotent stem cells (“NUCREM™ cells”) will express higher levels of TRT gene product. It is further envisioned that RA induced differentiated cells derived from said NUCREM™ cells will downregulate expression of the TRT gene product. Hence, the expression of relatively high levels of TRT gene product in a cell culture is indicative of a stem cell-like phenotype.

It is further envisioned that pluripotent stem cells, upon treatment with retinoic acid, will down regulate the expression of TRT and begin to express genes indicative of differentiating cells of various lineages. For example, Schuldiner et al., (2000) demonstrated the increased expression of tissue specific lineage markers in cultures of human embryonic stem cells that have been treated with retinoic acid. Those lineage specific markers include brain-specific neurofilament (ectodermal), heart-specific cardiac actin (mesodermal) and liver-specific α1-antitrypsin (endodermal).

RNA was extracted from cultures of (1) human adult keratinocytes, (2) human keratinocytes after 5-aza-2′-deoxycytidine and trichostatin A treatment, (3) in vitro-derived adult pluripotent stem cells of the present invention (“NUCREM™ cells”) after Tat-cyclin B treatment, and (4) NUCREM™ cells treated with retinoic acid as described in example 3. The extraction of RNA was carried out using the Perfect RNA™ Eukaryotic Kit (Eppendorf AG, Hamburg, Del.), which employs a chaotropic guanidine isothiocyanate solution for cell lysis and RNase inactivation and a proprietary RNA binding matrix, according to the manufacturer's instructions. The extracted RNA was dissolved in RNase-free water provided in the Perfect RNA™ Eukaryotic Kit.

RT-PCR was performed using the QIAGEN® OneStep RT-PCR Kit (Qiagen Inc., Valencia, Calif.) according to the manufacturer's instructions. PCR amplification was preformed using the following protocol: 94° C. for 1 min., 55° C. for 1 min., 72° C. for 1 min., for 45 cycles. The oligonucleotide primers and used to detect the following gene products (mRNAs) are described in Table 1: human telomerase (“TRT”), neurofilament heavy chain (“NF”), α1-antitrypsin (“αAT”) and cardiac actin (“cACT”). To control for the quality of the extracted RNA and to serve as an internal quantification marker, human glyceraldehyde 3-phosphate dehydrogenase (“GAPDH”) oligonucleotide primers were included in the RT-PCR reaction.

Table 1: RT-PCR primer sets and cycling parameters.

| | |
|---|---|
| GENE | PRIMER SET |

| | | |
|---|---|---|
| GAPDH | 5′-GGGGAGCCAAAAGGGTCATCATCT-3′ | (SEQ ID NO: 2) |
| | 5′-GACGCCTGCTTCACCACCTTCTTG-3′ | (SEQ ID NO: 3) |
| TRT | 5′-CGGAGGTCATCGCCAGCATCATCA-3′ | (SEQ ID NO: 4) |
| | 5′-GTCCCGCCGAATCCCCGCAAACAG-3′ | (SEQ ID NO: 5) |
| NF | 5′-TGAACACAGACGCTATGCGCTCAG-3′ | (SEQ ID NO: 6) |
| | 5′-CACCTTTATGTGAGTGGACACAGAG-3′ | (SEQ ID NO: 7) |
| αAT | 5′-AGACCCTTTGAAGTCAAGGACACCG-3′ | (SEQ ID NO: 8) |
| | 5′-CCATTGCTGAAGACCTTAGTGATGC-3′ | (SEQ ID NO: 9) |
| cACT | 5′-TCTATGAGGGCTAGCCTTTG-3′ | (SEQ ID NO: 10) |
| | 5′-CCTGACTGGAAGGTAGATGG-3′ | (SEQ ID NO: 11) |

The RT-PCR products were run out on a 2% agarose gel, which was then stained with ethidium bromide. The intensity of the resultant DNA bands were quantified using the PHORETIX™ TotalLab densitometry software package developed by Nonlinear USA (Durham, N.C.). To determine the approximate relative percent change in the expression of TRT, NF, αAT and cACT in each of the experimental groups relative to the untreated keratinocytes, the following equation was applied (eq. 1):

x=([(a′/b′)/(a/b)]−1)°100%, Eq. 1:

wherein x is the relative percent change in expression of the gene of interest, b is the intensity of the GAPDH band in untreated keratinocytes, b′ is the intensity of the GAPDH band obtained from the experimental cells, a is the intensity of the gene-of-interest band obtained from the untreated keratinocytes, and a′ is the intensity of the gene-of-interest band obtained from the experimental cells.

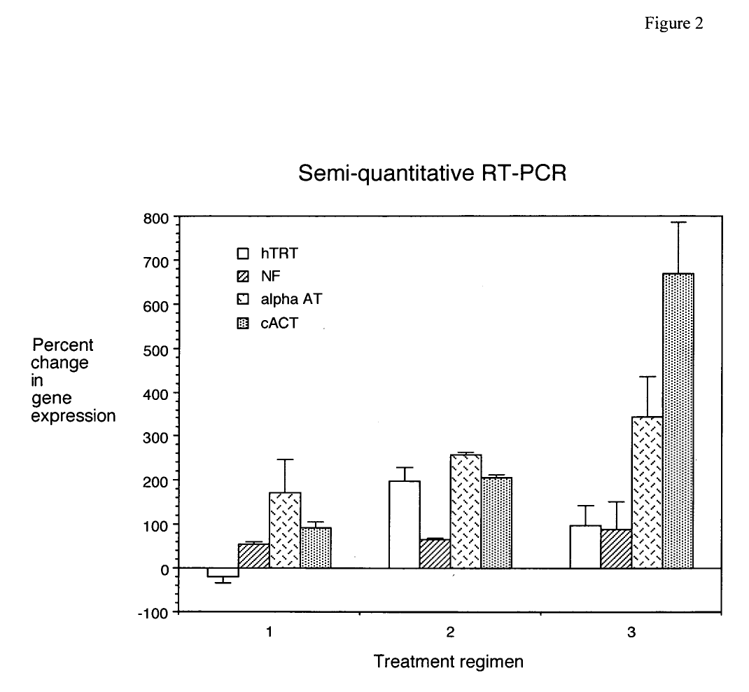

The results of three independent experiments are presented in FIG. 2. The vertical axis depicts the percent change in levels of gene expression relative to untreated keratinocytes. The error bars represent the standard error of the mean for three independent experiments. The horizontal axis depicts individual treatment regimens. Treatment regimen number 1 is human keratinocytes treated with 5-aza-2′-deoxycytidine and trichostatin A. Treatment regimen 2 is human keratinocytes treated with 5-aza-2′-deoxycytidine, trichostatin A and Tat-cyclin B (these cells are considered to be the in vitro derived adult pluripotent stem cells of the present invention (“NUCREM™ cells”). Treatment regimen 3 is NUCREM™ cells treated with retinoic acid as described in example 3.

It is important to note that the expression levels of TRT were increased by approximately 198% (±31%) in NUCREM™ cells compared to untreated keratinocytes. Upon subsequent treatment with retinoic acid, the levels of expression of TRT decreased approximately 50% (FIG. 2). These results are to be expected and indicate that the NUCREM™ cells of the instant invention have acquired the stem cell characteristic of telomerase gene expression.

Furthermore, upon treating NUCREM™ cells with retinoic acid, increased levels of expression of NF [increased by 89% (±45%)], αAT [increased by 343% (±94%)] and cACT [increased by 667% (±118%)] were observed. These results indicate that NUCREM™ cells have the capacity to give rise to cells of ectodermal, endodermal and mesodermal origin, respectively. Thus, the NUCREM™ cells of the present invention are pluripotent.

### EXAMPLE 5

### Further Demonstration of Pluripotency of In Vitro-Derived Pluripotent Stem Cells (“NUCREM™ Cells”)

It is envisioned that in vitro-derived adult pluripotent stem cells (“NUCREM™ cells”) of the present invention are cultured for 5 days on plastic petri dishes in ES cell medium without LIF and bFGF to induce formation of embryoid bodies. The embryoid bodies are collected by slow speed centrifugation. RNA is extracted from the embryoid bodies and RT-PCR is performed as described in example 4. PCR oligonucleotide primer pairs are designed using the following cDNA sequences found in the public database: muscle specific actin, Accession No. NM_{—}001615; desmin, Accession No. NM_{—}001927; CD34, Accession No. NM_{—}001773; alpha-fetoprotein, Accession No. NM_{—}001134. GAPDH and Oct-4 primer pairs are used as positive and negative controls, respectively. The down-regulation of Oct-4 expression and up-regulation of any or all of the differentiation markers described herein, demonstrate the ability of the in vitro-derived adult pluripotent stem cells (“NUCREM™ cells”) to differentiate along specific developmental pathways.

### EXAMPLE 6

### Differentiation of NUCREM™ Cells Into Insulin-Secreting Cells and Treatment of Humans Suffering from Diabetes

It is envisioned that the present invention may provide a source of pancreatic islet-like cells for the treatment of diabetes. The following protocol may be followed to generate implantable autologous cells programmed to produce glucose-responsive insulin-secreting cells. Expanded cultures of NUCREM™ cells of the present invention are plated into serum free medium to enrich for nestin-positive cells (see Lumelsky, 2001). The nestin-positive cells are then sub-subcultured and expanded for 6 to 7 days in serum-free N2 media supplemented with 1 μg/ml laminin, 10 ng/ml bFGF, 500 ng/ml N-terminal fragment of murine or human SHH (sonic hedge hog) 100 ng/ml FGF8 and B27 media supplement, as described in Lee et al. (2000) and Lumelsky (supra), which are herein incorporated by reference. After the nestin-positive cells are expanded, the growth factors (FGF, SHH) are removed from the media and nicotinamide is added to the media at a final concentration of 10 mM, to promote the cessation of cell proliferation and induce the differentiation of insulin-secreting cells. After approximately 6 days of growth factor starvation, aggregates of insulin-secreting cells are formed (islet-like cell clusters).

To treat human patients suffering from diabetes, it is envisioned that the insulin-secreting cells may be grafted subcutaneously into the patient, wherein the cells are either encapsulated in a polymer matrix or non-encapsulated. It is important to note that the NUCREM™ cells used to treat the patient were preferentially derived from said patient according to the invention. A therapeutic amount of insulin-secreting cells are implanted in the patient subcutaneously. The skilled practitioner may determine a therapeutic amount based upon the age, weight and general health of the patient and the amount of insulin secreted by said insulin-secreting cells in response to glucose administration. Blood glucose levels of the patient are monitored on a regular basis and the amount of implanted cells are adjusted accordingly.

What is claimed is:

1. A method of reprogramming a keratinocyte comprising treating a keratinocyte with a first agent, which promotes the demethylation of a nucleic acid, and a second agent, which inhibits the deacetylation of a histone protein, such that a reprogrammed cell is produced upon treating the keratinocyte with the first agent and the second agent, wherein the reprogrammed cell expresses a telomerase gene product and is capable of expressing a gene product which is not expressed by a keratinocyte.

2. The method of claim 1 wherein the first agent is a 5-aza-2′-deoxycytidine.

3. The method of claim 1 wherein the second agent is a trichostatin A.

4. The method of claim 1 wherein the keratinocyte is a human keratinocyte.

5. The method of claim 1 wherein the keratinocyte is treated with a third agent, which promotes the arrest of cells in metaphase.

6. The method of claim 5 wherein the third agent is a Tat-cyclin B.

7. The method of claim 6 wherein the gene product which is not expressed by a keratinocyte is selected from the group consisting of a neurofilament, a cardiac actin and an alpha-antitrypsin.

8. The method of claim 6 wherein the gene product which is not expressed by a keratinocyte is a cardiac actin.

9. The method of claim 5 wherein the keratinocyte is treated with a fourth agent, which promotes cell differentiation.

10. The method of claim 9 wherein the fourth agent is a retinoic acid.

11. The method of claim 10 wherein the reprogrammed cell expresses a gene product selected from the group consisting of a neurofilament, a cardiac actin and an alpha-antitrypsin.

12. The method of claim 10 wherein the reprogrammed cell expresses a cardiac actin gene product.

13. An in vitro derived cell which is capable of expressing a gene product which is not expressed by a naturally occurring keratinocyte, wherein the in vitro derived cell is produced by treating a keratinocyte in vitro with a first agent, which promotes the demethylation of a nucleic acid, a second agent, which inhibits the deacetylation of a histone protein, and a third agent, which promotes the arrest of cells in metaphase.

14. The in vitro derived cell of claim 13 wherein the first agent is a 5-aza-2′-deoxycytidine, the second agent is a trichostatin A and the third agent is a Tat-cyclin B.

15. The in vitro derived cell of claim 14 wherein the gene product which is not expressed by a naturally occurring keratinocyte is selected from the group consisting of neurofilament, cardiac actin and alpha-antitrypsin.

16. An in vitro derived cell which expresses a gene product, which is not expressed by a naturally occurring keratinocyte, wherein the in vitro derived cell is produced by treating a keratinocyte in vitro with a first agent, which promotes the demethylation of a nucleic acid, a second agent, which inhibits the deacetylation of a histone protein, a third agent, which promotes the arrest of cells in metaphase, and a fourth agent, which promotes cell differentiation.

17. The in vitro derived cell of claim 16 wherein the first agent is a 5-aza-2′-deoxycytidine, the second agent is a trichostatin A, the third agent is a Tat-cyclin B and the fourth agent is a retinoic acid.

18. The in vitro derived cell of claim 16 wherein the gene product, which is not expressed by a naturally occurring keratinocyte, is selected from the group consisting of neurofilament, cardiac actin and alpha-antitrypsin.

19. The in vitro derived cell of claim 16 wherein the gene product, which is not expressed by a naturally occurring keratinocyte, is a cardiac actin.


SEQUENCE LISTING (11 sequences)

SEQ ID NO: 1 - protein, 408 aa, Homo sapiens
EFGRKKRRQRMALLRRPTVSSDLENIDTGVNSKVKSHVTIRRTVLEEIGNRVTTRAAQVAKKAQNTKVPVQPTKTTNVNKQLKPTASVKPVQMEKLAPKGPSPTPEDVSMKEENLCQAFSDALLCKIEDIDNEDWENPQLCSDYVKDIYQYLRQLEVLQSINPHFLDGRDINGRMRAILVDWLVQVHSKFRLLQETLYMCVGIMDRFLQVQPVSRKKLQLVGITALLLASKYEEMFSPNIEDFVYITDNAYTSSQIREMETLILKELKFELGRPLPLHFLRRASKAGEVDVEQHTLAKYLMELTLIDYDMVHYHPSKVAAAASCLSQKVLGQGKWNLKQQYYTGYTENEVLEVMQHMAKNVVKVNENLTKFIAIKNKYASSKLLKISMIPQLNSKAVKDLASPLIGRS

SEQ ID NO: 2 - DNA, 24 bp, Homo sapiens
ggggagccaaaagggtcatcatct

SEQ ID NO: 3 - DNA, 24 bp, Homo sapiens
gacgcctgcttcaccaccttcttg

SEQ ID NO: 4 - DNA, 24 bp, Homo sapiens
cggaggtcatcgccagcatcatca

SEQ ID NO: 5 - DNA, 24 bp, Homo sapiens
gtcccgccgaatccccgcaaacag

SEQ ID NO: 6 - DNA, 24 bp, Homo sapiens
tgaacacagacgctatgcgctcag

SEQ ID NO: 7 - DNA, 25 bp, Homo sapiens
cacctttatgtgagtggacacagag

SEQ ID NO: 8 - DNA, 25 bp, Homo sapiens
agaccctttgaagtcaaggacaccg

SEQ ID NO: 9 - DNA, 25 bp, Homo sapiens
ccattgctgaagaccttagtgatgc

SEQ ID NO: 10 - DNA, 20 bp, Homo sapiens
tctatgagggctagcctttg

SEQ ID NO: 11 - DNA, 20 bp, Homo sapiens
cctgactggaaggtagatgg